(19) United States
(12) Patent Application Publication
(10) Pub. No.: US 2025/0179421 A1
(43) Pub. Date: Jun. 5, 2025

(54) METHOD AND COMPOSITION FOR INHIBITING MELANOIDIN FORMATION IN A YEAST PROPAGATION PROCESS

(71) Applicant: BL Technologies, Inc., Minnetonka, MN (US)

(72) Inventor: Michael Raab, Minnetonka, MN (US)

(21) Appl. No.: 18/698,893

(22) PCT Filed: Nov. 2, 2022
(86) PCT No.: PCT/US2022/079159

§ 371 Date: Apr. 5, 2024

Related U.S. Application Data
(60) Provisional application No. 63/278,908, filed on Nov. 12, 2021.

(51) Int. Cl. C12N 1/16; C12R 1/645

(52) U.S. Cl. CPC C12N 1/165; C12R 2001/645

(57) ABSTRACT

A method for inhibiting melanoidin formation in a yeast propagation process, the method provides for adding a treatment composition to a molasses substance present in a yeast propagation process, wherein the treatment composition comprises a lecithin. A composition for a yeast propagation process, the composition having a lecithin, and a molasses substance.

### CROSS REFERENCE TO RELATED APPLICATIONS

This application claims the priority benefit of U.S. Provisional Patent Application Ser. No. 63/278,908 filed Nov. 12, 2021, the entirety of which is incorporated herein by reference.

### FIELD OF INVENTION

The disclosed technology provides for a method and composition for a yeast propagation process, and more specifically, a method and composition for inhibiting melanoidin formation in a yeast propagation process.

### BACKGROUND OF THE INVENTION

Industrial propagation of yeast is generally accomplished with use of abundantly available and cheap agricultural and industrial wastes, mainly molasses, by successive submerged fermentations. After fermentation, the yeast biomass is harvested and may be subjected to downstream processing steps like washing, cell disruption, protein extraction and purification. Industrial yeast production generally involves the following stages: propagation (involving a number of fermentation processes), harvesting, concentration and/or drying, packaging and shipment.

In the commercial product of yeast, molasses (a by-product of the refining of sugar beets and/or sugar cane, and of which contains fermentable sugars) is most often used as feed for yeast growth in fermentations, wherein the yeast growth and resulting yeast cream is eventually washed and filtered. Prior to being used as yeast fermentation feed, the molasses must be sterilized to kill any bacteria present.

However, during this operation, melanoidin compounds (i.e. Maillard reaction products) are formed and thus are included in the existing molasses. The presence of these melanoidin compounds produce undesirable dark colored yeast in downstream yeast propagation and washing steps. The water waste obtained during washing contains these caramelized sugars (thus leading to dark, almost black-colored yeast), which is later concentrated using evaporation units. The presence of these caramelized sugars leads to increased scale and fouling deposition, thus requiring cleaning of the evaporators, and has a significant impact on the energy used for evaporation in the yeast propagation process.

Thus, what is needed in the art is a method and composition to overcome the challenges presented with the formation of melanoidin compounds in a yeast propagation process.

### SUMMARY OF THE INVENTION

The disclosed technology provides for a method and composition for a yeast propagation process, and more specifically, a method and composition for inhibiting melanoidin formation in a yeast propagation process

In one aspect of the disclosed technology, a method for inhibiting melanoidin formation in a yeast propagation process is provided. The method comprising: adding a treatment composition to a molasses substance present in a yeast propagation process, wherein the treatment composition comprises a lecithin.

In some embodiments, the lecithin comprises a soy lecithin, a non-soy lecithin, a sunflower lecithin, an egg yolk lecithin, or combination thereof.

In some embodiments, the treatment composition further comprises a surfactant. In some embodiments, the molasses substance is obtained from sugar cane, sugar beets, sorghum, or a combination thereof.

In some embodiments, the yeast propagation process comprises the formation of melanoidin compounds. In some embodiments, the yeast propagation process comprises a molasses sterilization step, and/or a yeast fermentation step.

In some embodiments, the treatment composition is added to the molasses substance prior to the yeast fermentation step. In some embodiments, the treatment composition is added to the molasses substance prior to the molasses sterilization step. In some embodiments, the treatment composition inhibits the formation of melanoidin compounds present during the sterilization step.

In some embodiments, from about 1 ppm to about 1000 ppm of the treatment composition is provided to the molasses substance. In some embodiments, from about 25 ppm to about 500 ppm of the treatment composition is provided to the molasses substance.

In yet another aspect of the disclosed technology, a composition for a yeast propagation process is provided. The composition comprising: a lecithin, and a molasses substance.

In some embodiments, the lecithin comprises a soy lecithin, a non-soy lecithin, a sunflower lecithin, an egg yolk lecithin, or combination thereof.

In some embodiments, the molasses substance is obtained from sugar cane, sugar beets, sorghum, or a combination thereof.

In some embodiments, the composition further comprises a surfactant. In some embodiments, the surfactant is a non-ionic surfactant. In some embodiments, the surfactant comprises sorbitan fatty acid esters, ethoxylated mono and diglycerides, and/or ethoxylated soy bean oil.

In some embodiments, the ratio of lecithin to surfactant is about 50:50 to about 100:0. In some embodiments, the composition further comprises polyethylene glycol.

### DETAILED DESCRIPTION OF EXEMPLARY EMBODIMENTS

The disclosed technology provides for a method and composition for a yeast propagation process, and more specifically, a method and composition for inhibiting melanoidin formation in a yeast propagation process.

As previously described, melanoidin compounds are Maillard reaction products that are formed during certain stages within the yeast propagation process. During the Maillard reaction, in an early stage, a reducing sugar, condenses with a compound possessing a free amino group to give a condensation product (e.g. N-substituted glycosilamine, which rearranges to form the Amadori rearrangement product (ARP)), and which is subsequently degraded into a variety of compounds (e.g. carbonyl groups, dicarbonyl compounds) that are highly reactive and take part in further reactions. Subsequently, in an advanced stage, a range of reactions take place (including cyclisations, dehydrations, retroaldolisations, rearrangements, isomerisations and further condensations), which ultimately, in a final stage, lead to the formation of brown nitrogenous polymers and co-polymers, known as melanoidins.

It is believed that the disclosed method and treatment composition described herein interact with the sugars, proteins, and amino acids that are present in the yeast propagation process to reduce or interrupt the level of Maillard reactions that occur, and therefore, inhibit melanoidin formation. Inhibiting the melanoidin formation in this process would lead to (i) a decrease in water used to wash the yeast, (ii) a decrease in evaporator fouling due to melanoidins in the downstream process of yeast propagation, therefore prolonging the time between cleanings and reducing the energy cost needed for evaporation, as well as (iii) a reduction in water use, which lowers energy use with cleaner evaporators and extended production time.

The disclosed technology provides for a method for inhibiting melanoidin formation in a yeast propagation process. The method comprises adding a treatment composition to a molasses substance present in a yeast propagation process.

The treatment composition comprises a lecithin. In some embodiments, the lecithin comprises a soy lecithin, a non-soy lecithin, a sunflower lecithin, an egg yolk lecithin, or combination thereof. It should be understood by one skilled in the art that the lecithin can be obtained from any lecithin containing material. It is believed that the lecithin inhibits melanoidin formation by interacting with the amino compounds that are present in multiple places of the yeast propagation process.

In some embodiments, the treatment composition of the disclosed method further comprises a surfactant. It is believed that the surfactant aids the lecithin to disperse into the water phase with more ease, and will lower the surface tension of the mixture, thus, allowing better wetting (thereby greater contact) of the actives onto solid particles.

The treatment composition of the disclosed technology comprises a molasses substance. In some embodiments, the molasses substance is obtained from sugar cane, sugar beets, sorghum, or a combination thereof.

As previously described, the disclosed method is directed toward inhibiting the formation of melanoidin compounds that are present in a yeast propagation process. It should be understood that the conventional yeast propagation process includes a number of processing steps. In some embodiments, the yeast propagation process comprises a molasses sterilization step, and/or a yeast fermentation step.

In some embodiments, the treatment composition is added to the molasses substance prior to the yeast fermentation step, and in other embodiments, the treatment composition is added to the molasses substance prior to the molasses sterilization step. It is believed that it is during the molasses sterilization step, a majority of the sugars present suffer the Maillard reaction, and thus, produce the melanoidin compounds that are responsible for the undesirable dark colored yeast found during yeast propagation. In some embodiments, the treatment composition inhibits the formation of melanoidin compounds present during the sterilization step.

In some embodiments, from about 1 ppm to about 1000 ppm of the treatment composition is provided to the molasses substance. In other embodiments, from about 25 ppm to about 500 ppm of the treatment composition is provided to the molasses substance, and in other embodiments, from about 50 ppm to about 400 ppm of the treatment composition is provided to the molasses substance.

The disclosed technology further provides for a composition for a yeast propagation process. It is believed that the composition as described herein inhibits the melanoidin formation by interrupting the Maillard reactions present in a yeast propagation process.

The composition comprises a lecithin and a molasses substance. In some embodiments, the lecithin comprises a soy lecithin, a non-soy lecithin, a sunflower lecithin, an egg yolk lecithin, or combination thereof. In some embodiments, the molasses substance is obtained from sugar cane, sugar beets, sorghum, or a combination thereof.

In some embodiments, the composition further comprises a surfactant. In some embodiments, the surfactant is a non-ionic surfactant. In some embodiments, the surfactant comprises sorbitan fatty acid esters, ethoxylated mono and diglycerides, and/or ethoxylated soy bean oil.

In some embodiments, the ratio of lecithin to surfactant is about 50:50 to about 100:0. In some embodiments, the ratio of lecithin to surfactant is about 90:10 to about 65:35.

In some embodiments, the composition further comprises polyethylene glycol.

### Examples

The present technology will be further described in the following examples, which should be viewed as being illustrative and should not be construed to narrow the scope of the disclosed technology or limit the scope to any particular embodiments.

To demonstrate the ability of lecithin-based treatments of the present technology to reduce formation of dark coloration of yeast, a test was designed and carried out according to the following. A sample of molasses was treated with a lecithin-based composition at various concentrations. The molasses samples were then sterilized, and added to yeast samples that continued through the propagation process. After centrifugation, the yeast solids were evaluated and rated on a comparative visual scale.

The molasses samples were diluted to a concentration that consisted of 50% dissolved solids with a sugar content of 42° Bx. The samples were then treated with 0, 100, 200, and 400 ppm of a lecithin containing composition. These samples were then processed in the following process, as shown in Table 1.

TABLE 1

| | |
|---|---|
| Additive to molasses | 0, 100, 200, 400 ppm |
| Sterilization | 120° C., 3 hr. |
| Cool down | 2 hr |
| Inoculation to yeast | — |
| Fermentation | 24 hr |
| 1^{st} Centrifugation | 10 min |
| 2^{nd} Centrifugation of the solids | 10 min |

The solid portion of the samples was then evaluated visually and rated on a comparative 0-3 scale for the ability to inhibit dark coloration. The scale is shown as in Table 2 below.

TABLE 2

| Rating | Description |
|---|---|
| 0 | Poor, dominated by dark color |
| 1 | Moderate, approximately the same amount of |
| | dark and light color |
| 2 | Good, some dark color |
| 3 | Very good, very little dark color |

The results obtained are provided in Table 3 below.

TABLE 3

| Treatment Level | |
|---|---|
| ppm | Rating |
| 0 | 0 |
| 100 | 1 |
| 200 | 3 |
| 400 | 3 |

While embodiments of the disclosed technology have been described, it should be understood that the present disclosure is not so limited and modifications may be made without departing from the disclosed technology. The scope of the disclosed technology is defined by the appended claims, and all devices, processes, and methods that come within the meaning of the claims, either literally or by equivalence, are intended to be embraced therein.

1. A method for inhibiting melanoidin formation in a yeast propagation process, the method comprising:
adding a treatment composition to a molasses substance present in a yeast propagation process, wherein the treatment composition comprises a lecithin.

2. The method according to claim 1, wherein the lecithin comprises a soy lecithin, a non-soy lecithin, a sunflower lecithin, an egg yolk lecithin, or combination thereof.

3. The method according to claim 1, wherein the treatment composition further comprises a surfactant.

4. The method according to claim 1, wherein the molasses substance is obtained from sugar cane, sugar beets, sorghum, or a combination thereof.

5. The method according to claim 1, wherein the yeast propagation process comprises the formation of melanoidin compounds.

6. The method according to claim 1, wherein the yeast propagation process comprises a molasses sterilization step, and/or a yeast fermentation step.

7. The method according to claim 6, wherein the treatment composition is added to the molasses substance prior to the yeast fermentation step.

8. The method according to claim 6, wherein the treatment composition is added to the molasses substance prior to the molasses sterilization step.

9. The method according to claim 8, wherein the treatment composition inhibits the formation of melanoidin compounds present during the sterilization step.

10. The method according to claim 1, wherein from about 1 ppm to about 1000 ppm of the treatment composition is provided to the molasses substance.

11. The method according to claim 10, wherein from about 25 ppm to about 500 ppm of the treatment composition is provided to the molasses substance.

12. A composition for a yeast propagation process, the composition comprising:
a lecithin; and
a molasses substance.

13. The composition according to claim 12, wherein the lecithin comprises a soy lecithin, a non-soy lecithin, a sunflower lecithin, an egg yolk lecithin, or combination thereof.

14. The composition according to claim 12, wherein the molasses substance is obtained from sugar cane, sugar beets, sorghum, or a combination thereof.

15. The composition according to claim 12, further comprising a surfactant.

16. The composition according to claim 15, wherein the surfactant is a non-ionic surfactant.

17. The composition according to claim 16, wherein the surfactant comprises sorbitan fatty acid esters, ethoxylated mono and diglycerides, and/or ethoxylated soy bean oil.

18. The composition according to claim 15, wherein the ratio of lecithin to surfactant is about 50:50 to about 100:0.

19. The composition according to claim 15, further comprising polyethylene glycol.
